(19) United States
(12) Patent Application Publication
(10) Pub. No.: US 2008/0086180 A1
(43) Pub. Date: Apr. 10, 2008

(54) Techniques for gall bladder stimulation

(76) Inventors: Omry Ben-Ezra, Tel Aviv (IL); Tamir Ben-David, Tel Aviv (IL); Ehud Cohen, Ganei Tikva (IL)

Correspondence Address: COOPER & DUNHAM, LLP, 1185 AVENUE OF THE AMERICAS, NEW YORK, NY, 10036, US

(21) Appl. No.: 11/732,157

(22) Filed: Apr. 2, 2007

Related U.S. Application Data
(60) Provisional application No. 60/850,192, filed on Oct. 5, 2006.

(51) Int. Cl. A61N 1/00

(52) U.S. Cl. 607/40

(57) ABSTRACT

Apparatus for treating a patient is provided, including an electrode device, which is configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port. The apparatus further includes a control unit, which is configured to drive the electrode device to apply a current to the site, and to configure the current to induce weight loss of the patient. Other embodiments are also described.

### CROSS-REFERENCES TO RELATED APPLICATIONS

The present patent application claims the benefit of U.S. Provisional Patent Application 60/850,192, filed Oct. 5, 2006, entitled, “Gall bladder stimulation for treatment of obesity,” which is assigned to the assignee of the present application and is incorporated herein by reference.

### FIELD OF THE INVENTION

The present invention relates generally to treating patients by application of stimulation to selected tissue, and specifically to methods and apparatus for stimulating the gall bladder.

### BACKGROUND OF THE INVENTION

Obesity is an increasingly important health problem, causing morbidity and mortality as well as social and psychological discomfort. Conventional non-invasive techniques for treating obesity include dietary modification, physical exercise, and psychological counseling. Conventional surgical techniques include bariatric surgery, in which the stomach is surgically restricted or partially removed. Experimental approaches include the electrical stimulation of the stomach, intestine, or duodenum (see, for example, Xing J et al., cited below).

Bile is produced by the liver, and stored in the gall bladder. Contraction of the gall bladder is induced by the release of cholecystokinin (CCK) from the duodenum, in response to the ingestion of food, particularly food having a high fat content. The gall bladder contracts after a delay after the ingestion of the food. Normally, the delay is at least 30 minutes.

The neural and hormonal circuitry involved in gall bladder contraction is extensive, including vagal fibers (Liu C Y et al. [2004]; citation below), as well as other autonomic fibers (Ford G T et al. [1988]), and the action of various hormones, such as CCK.

The gall bladder is rich with sensory fibers (as evident from the acute pain associated with gall bladder inflammation). The gall bladder is connected to many reflex circuits. Some of the nerve fibers originating at the gall bladder join the vagus nerve, while other fibers connect at sympathetic plexi and ganglia.

CCK has been suggested as a potential agent for the treatment of obesity. CCK is a neuropeptide released by duodenal cells in response to fatty meal ingestion. CCK acts via two receptors, CCK-1 and CCK-2. CCK has been shown to induce satiety and reduce food intake in animal models (see, for example, Date Y et al., cited below). The satiety effect of CCK has been shown to be mediated by afferent fibers of the hepatic branch of the vagus nerve. Many CCK receptors and CCK-sensitive afferent fibers are located at the gall bladder.

Bariatric surgery has been found to have a favorable effect on diabetes (see, for example, del Amo D A et al., cited below).

PCT Publication WO 04/078252 to Karashurov, which is incorporated herein by reference, describes an implantable system for the treatment of human diseases by electric stimulation and/or electric blocking of the body tissues. In one embodiment, the system is used to treat a patient suffering from digestive and biliary disorders caused by gastric ulcer and dyskinesia of the bile duct. A chip is implanted into subcutaneous fat in the infraclavicular region. A gastric juice pH sensor is videolaparoscopically stitched to the stomach such that a first channel electrode is connected to sympathetic nerves of the stomach. A gall bladder bile sensor is stitched to the gall bladder wall such that a second chip channel electrode is connected to the gall bladder muscular wall. The chip is programmed so that sympathetic nerves of the stomach are stimulated every three hours to reduce the higher gastric juice pH, which is one of the reasons for the gastric ulcer. The gastric juice pH sensor is programmed so that nerve stimulation is stopped as soon as the gastric juice pH is reduced to 6. The channel connected to the gall bladder is programmed so that gall bladder contractions are induced during breakfast, lunch, and dinner, which results in bile inflow to the duodenum and improves digestion. The sensor stitched to the gall bladder is programmed so that the second channel responsible for stimulating gall bladder contractions is disconnected as soon as gall bladder is emptied. This creates conditions to facilitate healing of gastric ulcer and better digestion by means of programmed emptying of malfunctioning bile ducts.

US Patent Application Publication 2004/0172088 to Knudson et al., which is incorporated herein by reference, describes treatment of at least one of a plurality of disorders of a patient associated with vagal activity innervating at least one of a plurality of organs of the patient at an innervation site, by positioning a neurostimulator carrier within a body lumen of the patient. An electrode disposed on the carrier is positioned at a mucosal layer of the lumen. An electrical signal is applied to the electrode to modulate vagal activity by an amount selected to treat the disorder. The signal may be a blocking or a stimulation signal. The publication describes techniques for reestablishing the link between gastric emptying and pancreatic secretion delivery, thereby addressing the main pathology of this disease by shortening chyme residence time in the superior duodenum so that intestinal contents move into the distal digestive tract in a more normal manner. According to a first embodiment, this is done by stimulating the H+ ion receptors or by stimulation of the pancreas directly or via its parasympathetic innervation (preganglionic vagal nerves). As an additional alternative to pancreatic stimulation, the gall bladder can be stimulated to encourage bile movement into the duodenum. The bile can normalize the chyme to accelerate duodenal emptying. The gall bladder or bile duct can be stimulated indirectly via stimulation of the vagal nerve or directly stimulated by an electrode.

US Patent Application Publication 2005/0131485 to Knudson et al., which is incorporated herein by reference, describes a method for treating at least one disorder characterized at least in part by vagal activity. The method includes positioning an electrode at a body organ innervated by the vagus. An electrical signal is applied to the electrode to modulate vagal activity. The electrical signal is applied at a frequency in excess of 3,000 Hz for the signal to create a neural conduction block to the vagus with the neural conduction block selected to at least partially block nerve impulses on the vagus.

U.S. Pat. No. 6,571,127 to Ben-Haim et al., which is incorporated herein by reference, describes methods of increasing contractile force and/or the motility of a GI tract. A first method comprises selecting a portion of the GI tract and applying a non-excitatory electric field to the portion, which field increases the force of contraction at the portion. A second method comprises determining a timing of a returning wave in the GI tract and applying an electric field to at least a portion of the GI tract, which electric field reduces the response of the GI tract to the returning wave. Control of smooth muscles is especially employed in the gastrointestinal (GI) tract, the uterus, the bladder, endocrine glands, the gall bladder and blood vessels.

U.S. Pat. No. 5,256,640 to Peterson et al., which is incorporated herein by reference, describes nutritional supplements and methods of use of the supplements to maximally stimulate gall bladder contractions in patients undergoing rapid weight loss, to prevent the build-up of biliary sludge and/or gallstones.

PCT Publication WO 99/59666 to Wiener et al., which is incorporated herein by reference, describes methods and devices for selective delivery of therapeutic substances to specific histologic or microanatomic areas of organs. Introduction of the therapeutic substance into a hollow organ space (such as an hepatobiliary duct or the gall bladder lumen) at a controlled pressure, volume or rate allows the substance to reach a predetermined cellular layer (such as the epithelium or sub-epithelial space). The volume or flow rate of the substance can be controlled so that the intraluminal pressure reaches a predetermined threshold level beyond which subsequent subepithelial delivery of the substance occurs. Alternatively, a lower pressure is selected that does not exceed the threshold level, so that delivery occurs substantially only to the epithelial layer. Such site specific delivery of therapeutic agents permits localized delivery of substances (for example to the interstitial tissue of an organ) in concentrations that may otherwise produce systemic toxicity.

The following references, all of which are incorporated herein by reference, may be of interest:

US Patent Application Publication 2005/0038484 to Knudson et al.

US Patent Application Publication 2004/0167583 to Knudson et al.

U.S. Pat. No. 6,609,025 to Barrett et al.

U.S. Pat. No. 6,587,719 to Barrett et al.

US Patent Application Publication 2004/0024428 to Barrett et al.

US Patent Application Publication 2004/0039427 to Barrett et al.

U.S. Pat. Nos. 5,188,104 and 5,263,480 to Wernicke et al.

U.S. Pat. No. 5,540,730 to Terry, Jr. et al.

US Patent Application Publication 2005/0065575 to Dobak

U.S. Pat. No. 6,684,104 to Gordon et al.

U.S. Pat. No. 6,832,114 to Whitehurst et al.

Liu C Y et al., “Microinjection of glutamate into dorsal motor nucleus of the vagus excites gall bladder motility through NMDA receptor—nitric oxide—cGMP pathway,” Neurogastroenterol Motil 16(3):347-53 (2004)

Ford G T et al., “Inhibition of breathing associated with gall bladder stimulation in dogs,” Journal of Applied Physiology 65(1):72-79 (1988)

Pavlov, Conditioned Reflexes: An Investigation of the Physiological Activity of the Cerebral Cortex, London: Oxford University Press (1927)

Xing J et al., “Gastric electrical-stimulation effects on canine gastric emptying, food intake and body weight,” Obesity Research 11(1):41-47 (2003)

Date Y et al., “Peripheral interaction of ghrelin with cholecystokinin on feeding regulation,” Endocrinology 146(8):3518-3525 (2005)

del Amo D A et al., “Effect of vertical banded gastroplasty on hypertension, diabetes and dyslipidemia,” Obes Surg 12(3):319-23 (2002)

Hong J et al., “An ultrasound-driven needle-insertion robot for percutaneous cholecystostomy,” Phys Med Biol 49(3):441-55 (2004)

### SUMMARY OF THE INVENTION

In some embodiments of the present invention, apparatus and methods are provided for applying stimulation to a gall bladder site of a subject, as defined hereinbelow, and configuring the stimulation to cause weight loss of the subject. Such weight loss is typically caused by reduced intake of food and/or reduced absorption of ingested food, acutely and/or chronically.

In some embodiments of the present invention, the stimulation of the gall bladder site causes reduced food intake by inducing a feeling of satiety. Without wishing to be bound by any particular theory, the inventors hypothesize that the feeling of satiety is induced by one or more of the following physiological mechanisms:
the stimulation of the gall bladder site mimics the satiety-inducing effect of cholecystokinin (CCK). Many CCK receptors and CCK-sensitive afferent fibers are located in the vicinity of the gall bladder;
the stimulation of the gall bladder site causes gastric contraction, such as of an area of the stomach in a vicinity of the pyloric valve, or the pyloric valve itself. Such gastric contraction causes a feeling of satiety. The inventors hypothesize that the feeling of satiety is caused by the resulting increased gastric wall tension and/or delayed gastric emptying;
the stimulation of the gall bladder site induces a feeling of satiety because of the natural, lifelong association between gall bladder contraction and the feeling of satiety. The coordinated natural activity of gall bladder contractions at times of satiety causes a conditioned association between gall bladder contraction and the feeling of satiety;
the stimulation of the gall bladder site causes a reduction in food intake by stimulating increased and/or earlier release of bile from the gall bladder;
the stimulation of the gall bladder site causes the reduced food intake by some other indirect or direct pathway, such as those described hereinbelow; and/or
the stimulation of the gall bladder site causes a reduction in food intake by disturbing the naturally-occurring organized rhythmic activity of the stomach.

In some embodiments of the present invention, the stimulation of the gall bladder site causes reduced absorption of ingested food. Without wishing to be bound by any particular theory, the inventors hypothesize that the reduction in absorption is induced by one or more of the following physiological mechanisms:
the stimulation of the gall bladder site causes an increase in peristaltic motion of one or more distal organs of the gastrointestinal (GI) tract, such as the duodenum, jejunum, and/or ileum. Such increased peristaltic motion reduces the transit time of food, thereby reducing the absorption of food; and/or
the stimulation of the gall bladder site causes a reduction in absorption by stimulating increased and/or earlier release of bile from the gall bladder.

In some embodiments of the present invention, the gall bladder stimulation device comprises a coupling element that is configured to be placed around at least 270 degrees of at least one tubular gall bladder site, such as a neck of the gall bladder, a cystic duct, a hepatic duct, a common hepatic duct, a bile duct, and/or a common bile duct. The device also comprises at least one electrode that is coupled to the coupling element such that the at least one electrode is positioned in a vicinity of the at least one gall bladder site. For some applications, the coupling element is configured to be placed around the entire gall bladder site (i.e., 360 degrees of the site). For some applications, the coupling element comprises a cuff. For some applications, the coupling element is configured to be coupled to the site without suturing to any tissue of the subject.

In an experiment conducted by the inventors, a cuff electrode device was placed around a cystic duct of a dog near the neck of the gall bladder. Stimulation by the electrode device caused immediate, substantial gastric contraction, particularly of the pyloric area of the stomach.

In some embodiments of the present invention, gall bladder stimulation techniques described herein are used to treat a diabetic subject. The inventors hypothesize that such stimulation increases glucose tolerance of the subject. Alternatively or additionally, the stimulation treats diabetes by some other mechanism. For some applications, the gall bladder stimulation techniques described herein are used to treat a subject who is both overweight and diabetic. For some applications, the gall bladder stimulation techniques described herein are used for treating excessive eating, and/or another eating-related disorder.

The term “gall bladder site” is to be understood as including the gall bladder itself, including the neck thereof; the cystic duct; the hepatic duct, such as the common hepatic duct; the bile duct, such as the common bile duct; sensory fibers of the gall bladder; nerve plexuses or branches adjacent to the gall bladder, such as vagal nerve branches adjacent to the gall bladder; and the hepatic port. The term “gall bladder,” unless other specified, includes only the gall bladder itself.

There is therefore provided, in accordance with an embodiment of the present invention, apparatus for treating a patient, including:

an electrode device, configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

a control unit, configured to drive the electrode device to apply a current to the site, and to configure the current to induce weight loss of the patient.

In an embodiment, the gall bladder site includes the gall bladder, and the electrode device is configured to be placed in the vicinity of the gall bladder. Alternatively or additionally, the gall bladder site includes at least one site selected from the group consisting of: the neck of the gall bladder, and the cystic duct, and the electrode device is configured to be placed in the vicinity of the selected site.

For some applications, the control unit is configured to drive the electrode device to apply the current even in the absence of an indication of eating of the patient. For some applications, the control unit is configured to drive the electrode device to apply the current even in the absence of an indication of any voluntary or involuntary activity of the patient. Alternatively, the control unit is configured to drive the electrode device to apply the current responsively to an indication of eating of the patient.

In an embodiment, the control unit is not configured to configure the current to induce a contraction of the gall bladder that is sufficient to cause release of bile from the gall bladder. In other words, the control unit is not configured to induce the release of bile from the gall bladder, but such a release may nevertheless occur in some circumstances because of the applied current. For some applications, the control unit is not configured to configure the current to induce a contraction of the gall bladder that is sufficient to cause release of at least 2 ml of bile from the gall bladder during a 30-second period beginning at commencement of the application of the current.

For some applications, the control unit is configured to drive the electrode device to apply the current intermittently. For some applications, the control unit is configured to drive the electrode device to apply the current during at least 60 minutes per 48 hour period over at least 10 contiguous 48-hour periods. For some applications, the control unit is configured to drive the electrode device to apply the current during “on” periods alternating with “off” periods, each of the “on” periods on average has a duration of at least one second, and each of the “off” periods has a duration on average equal to at least 5 times the duration of the “on” periods on average. For some applications, the control unit is configured to receive a patient signal, and to drive the electrode device to apply the current responsively to the signal.

In an embodiment, the electrode device includes at least one electrode and a cuff coupled to the at least one electrode, and the cuff is configured to be placed around at least 270 degrees of the at least one site.

In an embodiment, the control unit is configured to configure the current to cause at least one effect selected from the group consisting of: gastric contraction, and delayed gastric emptying.

Typically, the electrode device is configured to be fully implanted in a body of the patient.

In an embodiment, the control unit is configured to configure the current to activate nervous tissue near or at the gall bladder site. For example, the nervous tissue may include at least one tissue selected from the group consisting of: nerve plexuses or branches adjacent to the gall bladder, and vagal nerve branches adjacent to the gall bladder, and the control unit is configured to configure the current to active the selected nervous tissue.

In an embodiment, the control unit is configured to induce the weight loss by inducing a feeling of satiety in the patient by driving the electrode device to apply the current. For some applications, the control unit is configured to induce the feeling of satiety by configuring the current to cause at least one effect selected from the group consisting of: gastric contraction, and delayed gastric emptying. Alternatively or additionally, the control unit is configured to induce the feeling of satiety by configuring the current to disturb naturally-occurring organized rhythmic activity of a stomach of the patient. Further alternatively or additionally, the control unit is configured to induce the feeling of satiety by configuring the current to cause at least one effect selected from the group consisting of: increased release of bile from the gall bladder than would occur in the absence of the current, and earlier release of bile from the gall bladder than would occur in the absence of the current.

In an embodiment, the control unit is configured to induce the weight loss by causing at least one effect by driving the electrode device to apply the current, the at least one effect selected from the group consisting of: a reduced absorption of ingested food, increased peristaltic motion of one or more organs of a gastrointestinal tract of the patient, and decreased transit time of food through a duodenum. For some applications, the effect includes the reduced absorption of the ingested food, and the control unit is configured to cause the reduced absorption by configuring the current to cause at least one effect selected from the group consisting of: increased release of bile from the gall bladder than would occur in the absence of the current, and earlier release of bile from the gall bladder than would occur in the absence of the current.

In an embodiment, the control unit is configured to configure the current to cause contraction of a stomach of the patient. For some applications, the control unit is configured to configure the current to cause contraction of the stomach in a vicinity of a pyloric valve. Alternatively or additionally, the control unit is configured to configure the current to cause contraction of a pyloric valve of the stomach.

There is further provided, in accordance with an embodiment of the present invention, apparatus for treating a patient, including:

a coupling element, configured to be placed around at least 270 degrees of at least one site of the patient selected from the group consisting of: a neck of a gall bladder, a cystic duct, a hepatic duct, a common hepatic duct, a bile duct, and a common bile duct; and

at least one electrode, coupled to the coupling element such that the at least one electrode is positioned in a vicinity of the at least one site when the coupling element is coupled to the at least one site.

For some applications, the coupling element is configured to be placed around 360 degrees of the at least one site.

For some applications, the coupling element is configured to be placed around at least 270 degrees of the at least one site without suturing to any tissue of the patient.

In an embodiment, the at least one site includes at least one site selected from the group consisting of: the neck of the gall bladder, and the cystic duct, and the coupling element is configured to be placed around at least 270 degrees of the selected site.

In an embodiment, the apparatus includes a control unit, configured to drive the at least one electrode to apply a current to the at least one site.

In an embodiment, the control unit is configured to configure the current to stimulate nervous tissue in a vicinity of the at least one site. For example, the nervous tissue may include at least one tissue selected from the group consisting of: nerve plexuses or branches adjacent to the gall bladder, and vagal nerve branches adjacent to the gall bladder, and the control unit is configured to configure the current to active the selected nervous tissue.

For some applications, the control unit is configured to configure the current to cause contraction of a stomach of the patient, such as in a vicinity of a pyloric valve.

For some applications, the control unit is configured to drive the at least one electrode to apply the current even in the absence of an indication of eating of the patient. For some applications, the control unit is configured to drive the at least one electrode to apply the current even in the absence of an indication of any voluntary or involuntary activity of the patient.

For some applications, the control unit is not configured to configure the current to induce a contraction of the gall bladder that is sufficient to cause release of bile from the gall bladder.

For some applications, the control unit is configured to drive the at least one electrode to apply the current intermittently. For some applications, the control unit is configured to drive the at least one electrode to applying the current during “on” periods alternating with “off” periods, each of the “on” periods on average has a duration of at least one second, and each of the “off” periods has a duration on average equal to at least 5 times the duration of the “on” periods on average.

In an embodiment, the control unit is configured to configure the current to induce weight loss of the patient. For some applications, the control unit is configured to induce the weight loss by inducing a feeling of satiety in the patient by driving the at least one electrode to apply the current.

In an embodiment, the control unit is configured to configure the current to treat diabetes of the patient.

For some applications, the control unit is configured to drive the at least one electrode to apply the current responsively to an indication of eating of the patient.

In an embodiment, the coupling element includes a cuff, which is configured to be placed around at least 270 degrees of the at least one site. For some applications, the cuff is configured to be placed around 360 degrees of the at least one site. For some applications, an inner diameter of the cuff is between 2 mm and 10 mm. For some applications, the cuff is configured to dynamically change a diameter thereof after being placed around at least 270 degrees of the at least one site. For some applications, the cuff includes an elastic material, configured to accommodate dynamic changes in a diameter of the at least one site. For some applications, the cuff is configured to be placed around at least 270 degrees of the at least one site during a laparoscopic surgical procedure. For some applications, the cuff is configured to be fully implanted in a body of the patient.

There is still further provided, in accordance with an embodiment of the present invention, apparatus for treating a patient, including:

a stimulator, configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

a control unit, configured to drive the stimulator to apply stimulation to the site during at least 60 minutes per 48 hour period over at least 10 contiguous 48-hour periods.

In an embodiment, the at least one gall bladder site includes the gall bladder, and the stimulator is configured to be placed in the vicinity of the gall bladder. Alternatively or additionally, the at least one gall bladder site includes the neck of the gall bladder, and the stimulator is configured to be placed in the vicinity of the neck of the gall bladder. Further alternatively or additionally, the at least one gall bladder site includes the cystic duct, and the stimulator is configured to be placed in the vicinity of the cystic duct.

For some applications, the control unit is configured to drive the stimulator to apply the stimulation even in the absence of an indication of eating of the patient. For some applications, the control unit is configured to drive the stimulator to apply the stimulation even in the absence of an indication of any voluntary or involuntary activity of the patient.

For some applications, the control unit is not configured to configure the stimulation to induce a contraction of the gall bladder that is sufficient to cause release of bile from the gall bladder.

For some applications, the control unit is configured to drive the stimulator to apply the stimulation intermittently.

In an embodiment, the stimulation includes mechanical stimulation, the stimulator includes a mechanical stimulator, and the control unit is configured to drive the stimulator to apply the mechanical stimulation to the site.

In an embodiment, the stimulation includes chemical stimulation, the stimulator includes a chemical stimulator, and the control unit is configured to drive the stimulator to apply the chemical stimulation to the site.

For some applications, the stimulator is configured to be fully implanted in a body of the patient.

In an embodiment, the stimulation includes electrical stimulation, the stimulator includes an electrical stimulator, and the control unit is configured to drive the stimulator to apply the electrical stimulation to the site.

In an embodiment, the electrical stimulator includes at least one electrode and a cuff coupled thereto, and the cuff is configured to be placed around at least 270 degrees of the gall bladder site.

For some applications, the control unit is configured to drive the electrical stimulator to apply the electrical stimulation during “on” periods alternating with “off” periods, each of the “on” periods on average has a duration of at least one second, and each of the “off” periods has a duration on average equal to at least 5 times the duration of the “on” periods on average.

For some applications, the control unit is configured to configure the electrical stimulation to activate nervous tissue near or at the gall bladder site. For example, the nervous tissue may include at least one tissue selected from the group consisting of: nerve plexuses or branches adjacent to the gall bladder, and vagal nerve branches adjacent to the gall bladder, and the control unit is configured to configure the electrical stimulation to active the selected nervous tissue.

There is additionally provided, in accordance with an embodiment of the present invention, apparatus for treating a patient, including:

a stimulator, configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

a control unit, configured to drive the stimulator to apply stimulation to the site even in the absence of an indication of eating of the patient.

For some applications, the control unit is configured to drive the stimulator to apply the stimulation even in the absence of a signal indicative of any voluntary or involuntary activity of the patient.

In an embodiment, the at least one gall bladder site includes the gall bladder, and the stimulator is configured to be placed in the vicinity of the gall bladder. Alternatively or additionally, the at least one gall bladder site includes the neck of the gall bladder, and the stimulator is configured to be placed in the vicinity of the neck of the gall bladder. Further alternatively or additionally, the at least one gall bladder site includes the cystic duct, and the stimulator is configured to be placed in the vicinity of the cystic duct.

For some applications, the control unit is not configured to configure the stimulation to induce a contraction of the gall bladder that is sufficient to cause release of bile from the gall bladder.

For some applications, the control unit is configured to drive the stimulator to apply the stimulation intermittently. For some applications, the control unit is configured to drive the stimulator to apply the stimulation during at least 60 minutes per 48 hour period over at least 10 contiguous 48-hour periods.

For some applications, the stimulator is configured to be fully implanted in a body of the patient.

In an embodiment, the stimulation includes mechanical stimulation, the stimulator includes a mechanical stimulator, and the control unit is configured to drive the stimulator to apply the mechanical stimulation to the site.

In an embodiment, the stimulation includes chemical stimulation, the stimulator includes a chemical stimulator, and the control unit is configured to drive the stimulator to apply the chemical stimulation to the site.

In an embodiment, the stimulation includes electrical stimulation, the stimulator includes an electrical stimulator, and the control unit is configured to drive the stimulator to apply the electrical stimulation to the site. For some applications, the electrical stimulator includes at least one electrode and a cuff coupled thereto, which cuff is configured to be placed around at least 270 degrees of the gall bladder site.

There is yet additionally provided, in accordance with an embodiment of the present invention, apparatus for treating a patient, including:

an electrode device, configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

a control unit, configured to drive the electrode device to apply a current to the site, and configure the current to cause contraction of a stomach of the patient.

For some applications, the control unit is configured to configure the current to cause contraction of the stomach in a vicinity of a pyloric valve. Alternatively or additionally, the control unit is configured to configure the current to cause contraction of a pyloric valve of the stomach.

For some applications, the control unit is configured to drive the electrode device to apply the current even in the absence of an indication of eating of the patient. For some applications, the control unit is configured to drive the electrode device to apply the current even in the absence of an indication of any voluntary or involuntary activity of the patient.

For some applications, the control unit is not configured to configure the current to induce a contraction of the gall bladder that is sufficient to cause release of bile from the gall bladder.

In an embodiment, the at least one gall bladder site includes the gall bladder, and the stimulator is configured to be placed in the vicinity of the gall bladder. Alternatively or additionally, the at least one gall bladder site includes the neck of the gall bladder, and the stimulator is configured to be placed in the vicinity of the neck of the gall bladder. Further alternatively or additionally, the at least one gall bladder site includes the cystic duct, and the stimulator is configured to be placed in the vicinity of the cystic duct.

For some applications, the control unit is configured to drive the electrode device to inhibit nervous tissue in a vicinity of the site.

For some applications, the control unit is configured to receive a patient signal, and to drive the electrode device to apply the current responsively to the signal.

In an embodiment, the electrical device includes at least one electrode and a cuff coupled thereto, which cuff is configured to be placed around at least 270 degrees of the gall bladder site. For some applications, the electrode device is configured to be fully implanted in a body of the patient.

In an embodiment, the control unit is configured to configure the current to activate nervous tissue in a vicinity of the site.

For example, the nervous tissue may include at least one tissue selected from the group consisting of: nerve plexuses or branches adjacent to the gall bladder, and vagal nerve branches adjacent to the gall bladder, and the control unit is configured to configure the current to active the selected nervous tissue.

There is also provided, in accordance with an embodiment of the present invention, apparatus for treating a patient, including:

a stimulator, configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

a control unit, configured to drive the stimulator to apply stimulation to the site, and not configured to configure the stimulation to induce a contraction of the gall bladder that is sufficient to cause release of bile from the gall bladder.

In an embodiment, the at least one gall bladder site includes the gall bladder, and the stimulator is configured to be placed in the vicinity of the gall bladder. Alternatively or additionally, the at least one gall bladder site includes the neck of the gall bladder, and the stimulator is configured to be placed in the vicinity of the neck of the gall bladder. Further alternatively or additionally, the at least one gall bladder site includes the cystic duct, and the stimulator is configured to be placed in the vicinity of the cystic duct.

For some applications, the control unit is configured to drive the stimulator to apply the stimulation even in the absence of an indication of eating of the patient. For some applications, the control unit is configured to drive the stimulator to apply the stimulation even in the absence of an indication of any voluntary or involuntary activity of the patient.

For some applications, the control unit is not configured to configure the stimulation to induce a contraction of the gall bladder that is sufficient to cause release of bile from the gall bladder.

In an embodiment, the stimulation includes mechanical stimulation, the stimulator includes a mechanical stimulator, and the control unit is configured to drive the stimulator to apply the mechanical stimulation to the site.

In an embodiment, the stimulation includes chemical stimulation, the stimulator includes a chemical stimulator, and the control unit is configured to drive the stimulator to apply the chemical stimulation to the site.

For some applications, the stimulator is configured to be fully implanted in a body of the patient.

In an embodiment, the stimulation includes electrical stimulation, the stimulator includes an electrical stimulator, and the control unit is configured to drive the stimulator to apply the electrical stimulation to the site. For some applications, the electrical stimulator includes at least one electrode and a cuff coupled thereto, and the cuff is configured to be placed around at least 270 degrees of the gall bladder site.

There is further provided, in accordance with an embodiment of the present invention, apparatus for treating a patient, including a mechanical stimulator, configured to be applied to at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port, and to mechanically stimulate the site.

In an embodiment, the at least one gall bladder site includes at least one site selected from the group consisting of: the gall bladder, the cystic duct, the common hepatic duct, and the common bile duct, and the mechanical stimulator is configured to be placed with a lumen of the selected site.

In an embodiment, the mechanical stimulator is configured to actively stimulate the at least one gall bladder site. For some applications, the mechanical stimulator is configured to cause at least one effect selected from the group consisting of: movement of the at least one gall bladder site, vibration of the at least one gall bladder site, stretching of the at least one gall bladder site, and contraction of the at least one gall bladder site.

In an embodiment, the mechanical stimulator is configured to passively stimulate the at least one gall bladder site. For some applications, the mechanical stimulator is shaped so as to define one or more surfaces configured to irritate the at least one gall bladder site.

There is still further provided, in accordance with an embodiment of the present invention, a method for treating a patient, including:

identifying that the patient may benefit from weight loss; and

responsively to the identifying, inducing the weight loss by applying electrical stimulation to at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port.

In an embodiment, applying the stimulation includes identifying that the electrical stimulation is suitable for activating nervous tissue in a vicinity of the gall bladder site. For example, the nervous tissue may include at least one tissue selected from the group consisting of: nerve plexuses or branches adjacent to the gall bladder, and vagal nerve branches adjacent to the gall bladder, and applying the stimulation includes identifying that the electrical stimulation is suitable for activating the selected nervous tissue.

There is additionally provided, in accordance with an embodiment of the present invention, a method for treating a patient, including:

placing a coupling element around at least 270 degrees of at least one site of the patient selected from the group consisting of: a neck of a gall bladder, a cystic duct, a hepatic duct, a common hepatic duct, a bile duct, and a common bile duct, such that at least one electrode coupled to the coupling element is positioned in a vicinity of the at least one site; and

applying an electrical current to the at least one site using the at least one electrode.

There is yet additionally provided, in accordance with an embodiment of the present invention, a method for treating a patient, including:

placing a stimulator in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

driving the stimulator to apply stimulation to the site during at least 60 minutes per 48 hour period over at least 10 contiguous 48-hour periods.

There is also provided, in accordance with an embodiment of the present invention, a method for treating a patient, including:

placing a stimulator in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

driving the stimulator to apply stimulation to the site even in the absence of an indication of eating of the patient.

There is further provided, in accordance with an embodiment of the present invention, a method for treating a patient, including:

applying a current to a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

configuring the current to cause contraction of a stomach of the patient.

There is still further provided, in accordance with an embodiment of the present invention, a method for treating a patient, including:

applying stimulation to a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

not configuring the stimulation to induce a contraction of the gall bladder that is sufficient to cause release of bile from the gall bladder.

There is also provided, in accordance with an embodiment of the present invention, a method for treating a patient, including:

applying a mechanical stimulator to at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and

mechanically stimulating the site with the mechanical stimulator.

There is further provided, in accordance with an embodiment of the present invention, a method for treating a patient, including:

identifying an electrical signal as being suitable for activating nervous tissue; and

applying the identified signal in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port.

In an embodiment, applying the identified signal includes applying the identified signal in the vicinity of at least one site selected from the group consisting of: the neck of the gall bladder, and the cystic duct.

For some applications, the nervous tissue includes at least one tissue selected from the group consisting of: nerve plexuses or branches adjacent to the gall bladder, and vagal nerve branches adjacent to the gall bladder, and identifying includes identifying that the electrical signal is suitable for activating the selected nervous tissue.

For some applications, applying the signal includes fully implanting an electrical stimulator in a body of the patient, and applying the signal using the electrical stimulator.

In an embodiment, the nervous tissue includes afferent nerve fibers, and identifying includes identifying that the signal is suitable for activating the afferent nerve fibers. For some applications, the nervous tissue includes nervous tissue of a vagus nerve, and identifying includes identifying that the signal is suitable for activating the vagal afferent nerve fibers.

There is still further provided, in accordance with an embodiment of the present invention, a method for treating a patient, including:

identifying that the patient suffers from diabetes; and

responsively to the identifying, treating the diabetes by applying electrical stimulation to at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port.

In an embodiment, applying the stimulation includes applying the stimulation to the gall bladder. Alternatively or additionally, applying the stimulation includes applying the stimulation to at least one site selected from the group consisting of: the neck of the gall bladder, and the cystic duct.

For some applications, applying the stimulation includes applying the stimulation even in the absence of an indication of eating of the patient. For some applications, applying the stimulation includes applying the stimulation even in the absence of an indication of any voluntary or involuntary activity of the patient.

For some applications, applying the stimulation does not include configuring the stimulation to induce a contraction of the gall bladder that is sufficient to cause release of bile from the gall bladder.

For some applications, applying the stimulation includes applying the stimulation intermittently. For some applications, applying the stimulation includes applying the stimulation during at least 60 minutes per 48 hour period over at least 10 contiguous 48-hour periods. For some applications, applying the stimulation includes applying the stimulation during “on” periods alternating with “off” periods, each of the “on” periods on average has a duration of at least one second, and each of the “off” periods has a duration on average equal to at least 5 times the duration of the “on” periods on average.

For some applications, applying the stimulation includes fully implanting an electrical stimulator in a body of the patient, and applying the stimulation using the electrical stimulator.

In an embodiment, applying the stimulation includes identifying that the electrical stimulation is suitable for activating nervous tissue in a vicinity of the gall bladder site. For example, the nervous tissue may include at least one tissue selected from the group consisting of: nerve plexuses or branches adjacent to the gall bladder, and vagal nerve branches adjacent to the gall bladder, and applying the stimulation includes identifying that the electrical stimulation is suitable for activating the selected nervous tissue.

The present invention will be more fully understood from the following detailed description of embodiments thereof, taken together with the drawings, in which:

### BRIEF DESCRIPTION OF THE DRAWINGS

FIG. 1 is a schematic illustration of a gall bladder stimulation system, in accordance with an embodiment of the present invention;

FIG. 2 is a simplified perspective illustration of an electrode device of the system of FIG. 1 in a slightly open position, in accordance with an embodiment of the present invention;

FIGS. 3A and 3B are cross-sectional views of a cuff of the electrode device of FIG. 2 in the plane A-A of FIG. 2 in open and closed positions, respectively, in accordance with an embodiment of the present invention;

FIG. 4 is a simplified perspective illustration of another configuration of the electrode device of the system of FIG. 1 in a slightly open position, in accordance with an embodiment of the present invention; and

FIG. 5 is a cross-sectional view of a cuff of the electrode device of FIG. 4 in the plane B-B of FIG. 4, in accordance with an embodiment of the present invention.

### DETAILED DESCRIPTION OF EMBODIMENTS

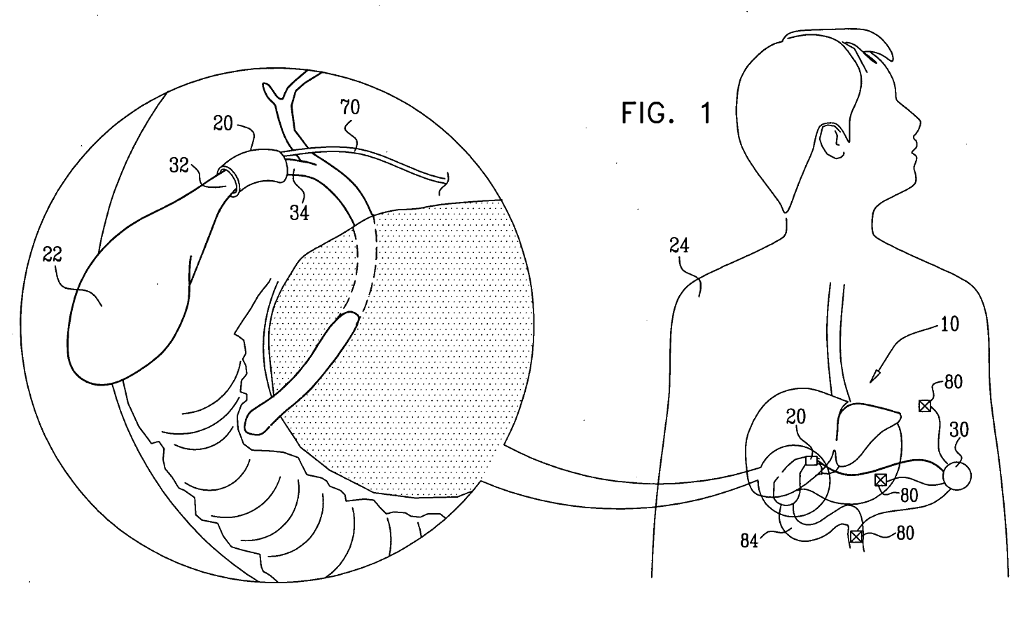

FIG. 1 is a schematic illustration of a gall bladder stimulation system 10, in accordance with an embodiment of the present invention. System 10 comprises an electrode device 20, which is configured to be applied to a site of a gall bladder 22 of a subject 24. For some applications, system 10 further comprises an implanted or external control unit 30, which is coupled to electrode device 20. Control unit 30 is configured to drive electrode device 20 to apply an electrical current to the site of the gall bladder. The location of implantation of control unit 30 shown in FIG. 1 is illustrative; the control unit may alternatively be implanted at other locations in the body, such as on the right side of the body in a vicinity of the gall bladder site.

The term “gall bladder site” is to be understood as including: gall bladder 22 itself, including a neck 32 thereof; a cystic duct 34; a hepatic duct, such as a common hepatic duct; a bile duct, such as a common bile duct; sensory fibers of gall bladder 22; nerve plexuses or branches adjacent to the gall bladder, such as vagal nerve branches adjacent to the gall bladder; and the hepatic port. For some applications, the gall bladder site includes a site within a lumen or wall of gall bladder 22, or a site outside the gall bladder in a vicinity thereof, such as a vicinity of a hepatic port, an inferior aspect of a right lobe of the liver, and gall bladder fossa. The term “gall bladder,” unless other specified, includes only gall bladder 22 itself, including the lumen, wall, and external surface thereof. The terms “stomach” and “gastric,” as used in the present application, including the claims, include within their scope the pyloric valve.

In an embodiment of the present invention, system 10, including control unit 30 and electrode device 20, is configured to be placed entirely within a duct (e.g., cystic duct 34, a hepatic duct, or a bile duct) and/or within the lumen of gall bladder 22.

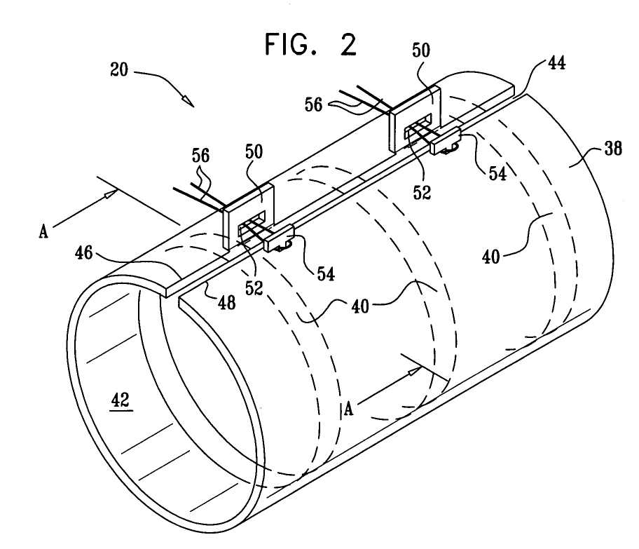

FIG. 2 is a simplified perspective illustration of electrode device 20 in a slightly open position, in accordance with an embodiment of the present invention. In this embodiment, electrode device 20 is configured to be applied to a tubular gall bladder site, such as gall bladder neck 32 and/or a duct of a gall bladder site, such as cystic duct 34; a hepatic duct, such as a common hepatic duct; or a bile duct, such as a common bile duct. The electrode device comprises a coupling element, which for some applications comprises a tubular insulating cuff 38, and one or more electrodes 40 fixed to and/or within the cuff. For some applications, electrodes 40 comprises ring electrodes, which are configured to apply a generally uniform current around the circumference of the duct/neck. For some applications, cuff 38 is sized and configured to encompass the duct/neck. Alternatively, the cuff is sized and configured to additionally encompass blood vessels, such as blood vessels supplying the gall bladder and overlying peritoneal lining, for applications in which the cuff is placed around the gall bladder neck or cystic duct. Typically, the cuff is configured to be placed around at least 270 degrees of the gall bladder site, such around the entire circumference of the gall bladder site (i.e., around 360 degrees of the site).

Cuff 38 typically comprises a flexible, resilient biocompatible material, such as silicone or polyurethane. For some applications, the cuff comprises an elastic material, which allows the cuff to accommodate dynamic changes in a diameter of the site around which the cuff is placed, such as the gall bladder neck or cystic duct. For some applications, the cuff comprises more than one material, for example, to provide better control of diameters, thicknesses, and/or strengths of various portions of the cuff. For some applications, electrodes 40 comprise platinum, a platinum alloy, titanium, and/or a titanium alloy. For some applications, the electrodes are recessed so as not to come in direct contact with the external tissue of the duct/neck.

Cuff 38 defines a central lumen 42 and, typically, a longitudinal slit 44. A first edge 46 of the cuff is brought in contact with a second edge 48 thereof in order to close the cuff around tubular tissue passing through lumen 42. Cuff 38 typically may be repeatedly opened and closed by a surgeon during a placement procedure, or to reposition or remove the cuff if necessary. At or near edge 46, cuff 38 comprises one or more flaps 50 set at an angle to the surface of the cuff, such as between 90 and 180 degrees, e.g., 90 degrees. Near the surface of the cuff, each of flaps 50 is shaped so as define an opening 52. At or near edge 48, cuff 38 comprises one or more anchoring elements 54, corresponding to openings 52. Typically, attached to each anchoring element 54 are one or more filaments 56, such as sutures or filaments made from silicone.

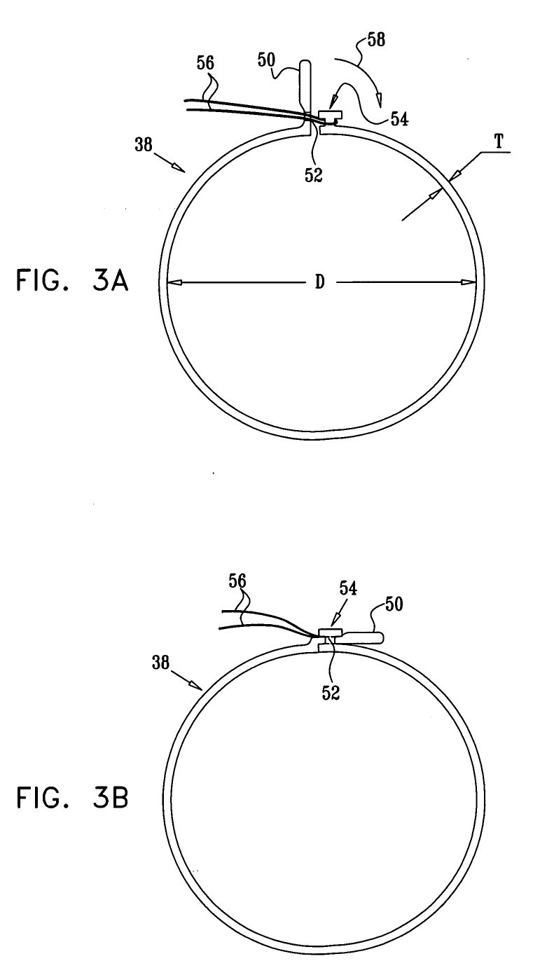

FIGS. 3A and 3B are cross-sectional views of cuff 38 in the plane A-A of FIG. 2 in open and closed positions, respectively, in accordance with an embodiment of the present invention. The surgeon threads each of filaments 56 through a corresponding one of openings 52, and then grasps the filament while simultaneously moving flap 50 in generally the opposite direction, i.e., in the direction indicated by an arrow 58 in FIG. 3A. As a result, anchoring element 54 is drawn through opening 52, thereby engaging the opening and closing cuff 38 around the tubular tissue of the gall bladder neck or cystic duct. Optionally, flap 50 is shaped so as to define an elongated tab, which a surgeon may grasp in order to assist in pulling the tab. Optionally, the surgeon clips off the filaments after the anchoring elements have engaged the openings.

The number of anchoring elements and openings which cuff 38 comprises depends on the length of the cuff. For example, cuff 38 may have a length of between about 4 and about 14 mm, and the cuff may comprise one, two, three, or four anchoring elements. A thickness T of the wall of cuff 38, at the wall's thinnest point, is typically between about 0.1 and 10 mm. An inner diameter D of the cuff is typically between about 2 mm and about 10 mm, e.g., between about 3 and about 5 mm.

For some applications, techniques described herein are used in combination with techniques described in U.S. patent application Ser. No. 10/529,149, filed Oct. 24, 2005, entitled, “Electrode assembly for nerve control,” and/or U.S. patent application Ser. No. 11/347,120, filed Feb. 2, 2006, entitled, “Electrode assembly for nerve control,” both of which are assigned to the assignee of the present application and are incorporated by reference. For example, electrode device 20 may utilize techniques described with reference to FIGS. 1A-5, 8-20, and/or 26-27 of the '120 application.

Alternatively, cuff 38 comprises one or more point electrodes fixed inside the cuff (configuration not shown). For example, techniques described in the '120 application with reference to FIG. 5 thereof may be used.

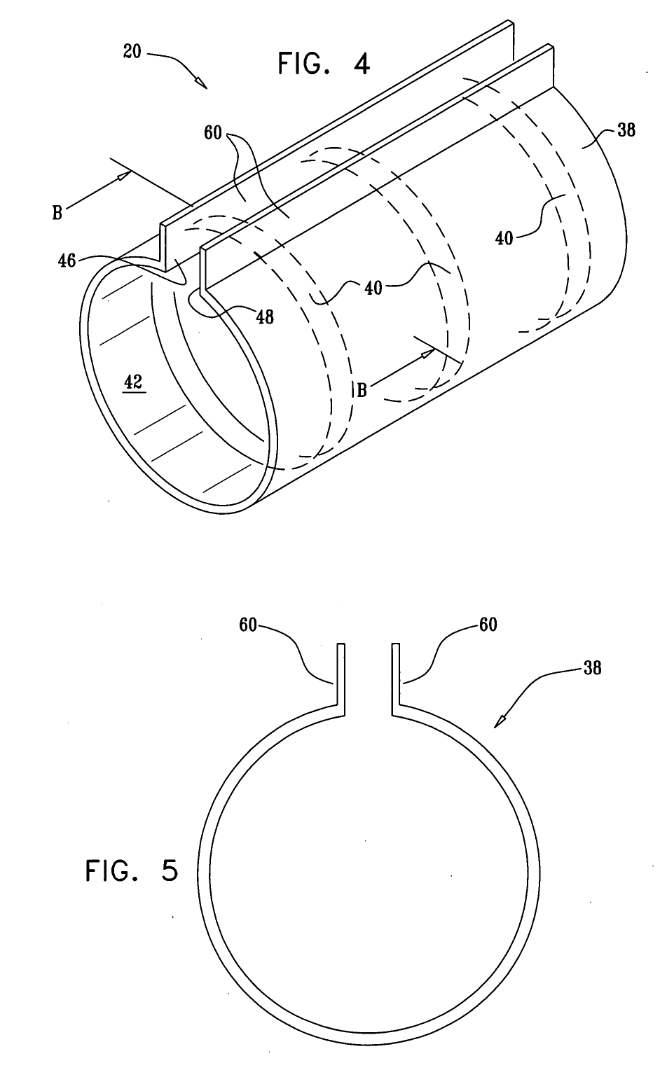

Reference is made to FIG. 4, which is a simplified perspective illustration of another configuration of electrode device 20 in a slightly open position, and to FIG. 5, which is a cross-sectional view of cuff 38 in the plane B-B of FIG. 4, in accordance with an embodiment of the present invention. This configuration is generally similar to that described hereinabove with reference to FIGS. 2 and 3A-B, except as noted below. At or near both edges 46 and 48, cuff 38 comprises one or more sets of tabs 60. For some applications, tabs 60 extend generally along the entire length of cuff 38, as shown in FIG. 4. For other applications, tabs 60 extend along only a portion of the length of cuff 38, in which case more than one pair of tabs may be provided (configuration not shown). To seal cuff 38 around the tubular site, the surgeon couples corresponding pairs of tabs 60 to one another, such as by stapling or suturing each of the tabs to a corresponding tab.

In an embodiment of the present invention, control unit 30 and/or electrode device 20 is configured to apply the electrical stimulation with a preferred activation direction, i.e., to generate more activation in one direction than in the other direction. For some applications, the greater activation direction is toward the body of gall bladder 22, while for other applications, the greater activation direction is toward the major papilla. For some applications, this preferred activation direction is achieved using unidirectional nerve stimulation techniques described in one or more of the applications incorporated by reference hereinbelow.

In an embodiment of the present invention, cuff 38 is configured to dynamically change its inner diameter after placement around the site. For some applications, the cuff is configured to have a larger inner diameter when no current is applied through the cuff, and a smaller inner diameter when current is applied. For some applications, such a dynamic change in diameter is effected using piezoelectric components, magnetic elements, or biological contracting elements.

In an embodiment of the present invention, electrode device 20 does not comprise a cuff. The electrodes of the electrode device are configured to be coupled to tissue of subject 24 such that the electrodes are positioned in a vicinity of a gall bladder site. For example, the electrodes may be in physical contact with gall bladder 22 (either an external surface of the gall bladder, or internal tissue of the gall bladder), or slightly remote from the gall bladder.

For some applications, electrode device 20 comprises at least one flexible patch electrode, which typically covers a large surface area, e.g., more than about 3 mm̂2. For some applications, the patch electrode has a plurality of conducting points, each of which is electrically coupled to control unit 30 separately or in groups. For some applications, a first portion of the conducting points are electrically coupled to a stimulating anode of control unit 30, and a second portion of the connecting points are electrically coupled to a stimulating cathode of the control unit.

In an embodiment of the present invention, control unit 30 configures the current applied by electrode device 20 to stimulate nervous tissue in a vicinity of the electrode device, such as by using nerve stimulation parameters known in the art. For some applications, the control unit configures the stimulation to activate nervous tissue in a vicinity of the site of application, while for other applications the control unit configures the stimulation to block nervous activity, for example by delivering high frequency stimulation. For example, blocking techniques may be used that are described in US Patent Application Publication 2005/0131485 to Knudson et al., which is incorporated herein by reference, mutatis mutandis.

Reference is again made to FIG. 1. For some applications, electrode device 20 is coupled to control unit 30 by at least one lead 70. Alternatively, the current is induced in the electrode device by means of magnetic induction. For some applications, control unit 30 comprises a power source, such as a battery. Alternatively or additionally, power is wirelessly transferred to control unit 30, or directly to electrode device 20, from a location outside the body of subject 24, such as using electromagnetic, ultrasound, or radiofrequency energy, or by induction. For some applications, the control unit is integrated into the electrode device, rather than provided as a separate component remote from the electrode device.

In an embodiment of the present invention, control unit 30 configures the current applied by electrode device 20 to cause weight loss of subject 24. Such weight loss is typically caused by reduced intake of food and/or reduced absorption of ingested food, acutely and/or chronically.

In some embodiments of the present invention, the stimulation of the gall bladder site causes reduced food intake by inducing a feeling of satiety. Without wishing to be bound by any particular theory, the inventors hypothesize that the feeling of satiety is induced by one or more of the following physiological mechanisms:
the stimulation of the gall bladder site mimics the satiety-inducing effect of cholecystokinin (CCK). Many CCK receptors and CCK-sensitive afferent fibers are located in the vicinity of the gall bladder;
the stimulation of the gall bladder site causes gastric contraction (i.e., a decrease in a cross-sectional area of the stomach), such as of an area of the stomach in a vicinity of the pyloric valve, the pyloric valve itself, or another portion of the stomach. Such gastric contraction causes a feeling of satiety. The inventors hypothesize that the feeling of satiety is caused by the resulting increased gastric wall tension and/or delayed gastric emptying;
the stimulation of the gall bladder site induces a feeling of satiety because of the natural, lifelong association between gall bladder contraction and the feeling of satiety. The coordinated natural activity of gall bladder contractions at times of satiety causes a conditioned association between gall bladder contraction and the feeling of satiety;
the stimulation of the gall bladder site causes a reduction in food intake by stimulating increased and/or earlier release of bile from the gall bladder;
the stimulation of the gall bladder site causes the reduced food intake by some other indirect or direct pathway, such as those described hereinbelow;
the stimulation of the gall bladder site causes a reduction in food intake by disturbing the naturally-occurring organized rhythmic activity of the stomach;
the stimulation of the gall bladder site causes a reduction in absorption by increased and/or earlier exocrine and/or endocrine release from the pancreas; and/or
the stimulation of the gall bladder site causes a shift in metabolism of absorbed food, reducing the formation of fat.

In some embodiments of the present invention, the stimulation of the gall bladder site causes reduced absorption of ingested food. Without wishing to be bound by any particular theory, the inventors hypothesize that the reduction in absorption is induced by one or more of the following physiological mechanisms:
the stimulation of the gall bladder site causes an increase in peristaltic motion of one or more distal organs of the gastrointestinal (GI) tract, such as the duodenum, jejunum, and/or ileum. Such increased peristaltic motion reduces the transit time of food, thereby reducing the absorption of food; and/or
the stimulation of the gall bladder site causes a reduction in absorption by stimulating increased and/or earlier release of bile from the gall bladder.

In an embodiment of the present invention, control unit 30 is configured to drive electrode device 20 to apply a fixed current without “on”/“off” cycles, e.g., at 1 Hz. Alternatively or additionally, the control unit is configured to apply the current at fixed intervals. For example, the control unit may apply intermittent stimulation including a pulse train having a frequency of between about 0.1 and about 20 Hz, e.g., about 5 Hz, a duration of each pulse of between about 0.1 and about 1 ms, e.g., about 0.5 ms, and a current of between about 0.1 to about 7 mA, e.g., about 5 mA. For some applications, such a train is applied during “on” periods alternating with “off” periods, wherein each of the “on” periods has a duration on average of between about 1 and about 30 seconds, e.g., 10 seconds, and each of the “off” periods has a duration on average of between about 5 and about 500 seconds, or a duration at least 5 times as long as each of the “on” periods.

In an embodiment of the present invention, control unit 30 is configured to drive electrode device 20 apply the current constantly. For example, the control unit may apply the constant stimulation including a pulse train having a frequency of between about 0.1 and about 20 Hz, e.g., about 5 Hz, a duration of each pulse of between about 0.1 and about 1 ms, e.g., about 0.5 ms, and a current of between about 0.1 to about 7 mA, e.g., about 5 mA.

For some applications, intrinsic physiological changes in gall bladder excitability cause the applied stimulation to excite the gall bladder at certain times, while failing to excite the gall bladder at other times. Nevertheless, the application of the stimulation generally increases the likelihood of gall bladder electrical activity, which causes an overall increase in gall bladder activity on average over time.

Furthermore, for some applications, the stimulation modulates the timing of spontaneous gall bladder activity, such that spontaneous activity occurs generally earlier after ingestion of food than would occur in the absence of the stimulation. Gall bladder contractions thus occur sooner after the ingestion of a meal than would occur in the absence of the stimulation.

In an embodiment of the present invention, control unit 30 configures the applied current to sensitize sensory fibers and reflex arcs originating in gall bladder 22. In this embodiment, the control unit typically configures the current to have a frequency between about 1 and about 20 Hz (e.g., about 3 Hz), a duty cycle of between about 10% and about 60%, a pulse duration of between about 0.1 and about 1 ms, e.g., about 0.5 ms, and a current of between about 0.1 and about 7 mA, e.g., about 5 mA. Sensitizing stimulation is recognizable by the delay in onset of effect. Typically, such sensitizing stimulation achieves its maximum effect only after several hours or several days of stimulation.

In an embodiment of the present invention, control unit 30 drives electrode device 20 to apply the stimulation during “high” stimulation periods alternating with “low” stimulation periods, i.e., the strength of the current during the “high” periods is greater than during the “low” periods. For some applications, the “high” stimulation periods are applied at fixed intervals (such as every 3 to 12 hours), or responsively to a signal from the subject and/or from a sensor (such as the sensors described herein). When the “high” stimulation is not applied, “low” stimulation is applied. For some applications, no stimulation is applied during the “low” periods.

Typically, the product of (a) the current times (b) the pulse duration times (c) the frequency times (d) the “on”/“off” ratio during the “high” periods is set to be on average at least 5 times the same product during the “low” periods.

For some applications, the “high” periods further include rhythmic cycling between “on” and “off” periods, imitating and/or eliciting gall bladder contraction. For example, such cycling may include “on” periods each of which has a duration on average of between about 0.1 and about 10 seconds, followed by “off” periods each of which has a duration on average of between about 1 and about 25 seconds. For some applications, the duration of each of the “high” periods is set to be on average between about 30 seconds and about 30 minutes.

For some applications, the applied pulses have a duration of up to about 2 seconds on average. Such long pulses are particularly appropriate for applications in which the stimulation is configured to induce gall bladder contraction.

In an embodiment of the present invention, the gall bladder site stimulation is configured to: (a) not cause contraction of the gall bladder sufficient to cause release bile from the gall bladder, while (b) causing activation of vagal afferent fibers, a reflex arc, and/or activation of other autonomic nervous tissue (together, “afferent feedback”). Such non-contraction-inducing stimulation is typically achieved by applying the stimulation to a location of the gall bladder site that does not cause contractions (e.g., a location on the gall bladder surface that does not cause contractions, or causes only a local contraction that is not sufficient to induce a release of bile from the gall bladder), but that still causes afferent feedback. Alternatively or additionally, such stimulation is achieved by setting one or more parameters of the stimulation, e.g., setting the stimulation to have a relatively low strength. Although in this embodiment the gall bladder stimulation is not configured to cause release of bile from the gall bladder, the stimulation may nevertheless sometimes cause the release of a small amount of bile, such as about 2 ml of bile during the first 30 seconds of each stimulation period.

For some applications, stimulation is applied when the gall bladder is not full of bile, such as when the gall bladder is substantially empty. Such stimulation causes no or only minimal release of bile (e.g., less than about 2 ml of bile during the 30 seconds of each stimulation period). Furthermore, such stimulation typically does not cause gall bladder contraction because the gall bladder generally does not contract when empty, even when stimulated. For some applications, the control unit applies the gall bladder stimulation responsively to finding that the gall bladder is empty and/or not full, such as by sensing a parameter indicative of such emptiness (e.g., impedance or stretch), or measuring a parameter indicative of such emptiness, such as time from ingestion of last meal.

In an embodiment of the present invention, gall bladder site stimulation is applied during one or more time periods not responsively to sensed or expected eating of the subject, i.e., even in the absence of an indication of eating of the subject. For example, the stimulation may be applied chronically, i.e., at least 130 minutes per day, such as at least 2.5 hours per day, over a period having a duration of at least 3 weeks, e.g., at least 3 months, or during at least 60 minutes per 48 hour period over at least 10 contiguous 48-hour periods. For some applications, the stimulation is applied even in the absence of an indication of any voluntary or involuntary activity of the subject. For some applications, such stimulation is configured to substantially not cause contraction of the gall bladder, while causing afferent feedback, as described hereinabove. Over time, such stimulation typically sensitizes the subject to vagal and/or other reflex arc activation, such that the subject becomes more sensitive to normal vagal and/or other reflex arc activation when the subject eats, or in response to stimulation of a gall bladder site using the techniques described herein. The inventors hypothesize that such sensitization over time causes a generally increased sensation of satiety, particularly during eating or application of the gall bladder site stimulation.

For some applications, gall bladder stimulation is configured to substantially not cause contraction of the gall bladder during a first time period, and to cause contraction of the gall bladder during a second time period. For example, such non-contraction-inducing stimulation may be applied chronically, as defined above, and such contraction-inducing stimulation may be applied during, prior to, and/or subsequent to food ingestion, as described hereinbelow.

For some applications, the control unit applies the stimulation in brief bursts separated on average by at least 20 seconds of no stimulation, to allow the induced GI activity to naturally fade out prior to the next stimulation. Each of the brief bursts typically has a duration of less than 2 seconds, e.g., less than 500 ms.

In an embodiment of the present invention, control unit 30 is configured to vary at least one parameter of stimulation randomly or quasi-randomly. For example, the parameter may include a time interval between sequential stimulation bursts, or the stimulation intensity. Such stimulation generally prevents the organized periodic activity necessary for normal and increased food intake. Normal gastric activity is typically characterized by organized contractions occurring at fixed intervals, such as three times per minute. Changes in gastric activity (such as frequency or force) are sensed and generally translated into a sensation of hunger. Disrupting the normal cycles of the stomach, in addition to the other effects described herein, interferes with this translation of the changes in gastric activity to a sensation of hunger.

In an embodiment of the present invention, gall bladder stimulation system 10 comprises a mechanical stimulator, instead of or in addition to electrode device 20. The mechanical stimulator is configured to be applied to a gall bladder site. For some applications, the mechanical stimulator is configured to be placed within the lumen of gall bladder 22, cystic duct 34, a common hepatic duct, or a common bile duct. Alternatively, the mechanical stimulator is configured to be placed within the body of the subject externally to these structures in a vicinity thereof.

For some applications, the mechanical stimulator is configured to apply passive stimulation, by irritating the site to which the stimulator is applied. For example, the stimulator may include rough surfaces, protrusions, or spikes. In some respects, the mechanical stimulator in these applications is similar to an intrauterine device (IUD), which mechanically irritates the uterus. For these applications, system 10 typically does not comprise control unit 30.

For other applications, the mechanical stimulator is configured to actively stimulate the site to which it is applied. For example, the stimulator may be configured to cause movement, vibration, stretching, or contraction of the site.

In an embodiment of the present invention, gall bladder stimulation system 10 comprises a chemical stimulator, instead of or in addition to the electrode device and/or the mechanical stimulator. The chemical stimulator is configured to be applied to a gall bladder site. For some applications, the chemical stimulator is configured to be placed within the lumen of gall bladder 22, cystic duct 34, a common hepatic duct, or a common bile duct. Alternatively, the chemical stimulator is configured to be placed within the body of the subject externally to these structures in a vicinity thereof. The chemical stimulator releases, such as by slow release, a chemical or biological substance that stimulates the gall bladder site. Such substances may include, for example, capsaicin, CCK, Ach, hydrogen ions or hydroxides, and/or free radicals.

For some applications, such mechanical and/or chemical stimulation is applied using applicable aspects of the electrical stimulation techniques described hereinabove, such as timing parameters, responsively to eating, not responsively to eating, and/or other techniques described herein.

Reference is again made to FIG. 1. In an embodiment the present invention, system 10 comprises one or more physiological sensors 80, which are configured to sense physiological parameters of subject 24. For some applications, one or more of these sensors is configured to be placed at a gastrointestinal (GI) tract location, such as a stomach 82 or an intestine 84, and to sense a GI tract parameter, such as electrical activity of the GI tract, stretching of the GI tract, or inner flow. Alternatively or additionally, one or more of sensors 80 is configured to be placed elsewhere in or on the body of subject 24, and to detect another parameter of the subject, such as blood glucose level.

In an embodiment of the present invention, control unit 30 is configured to drive electrode device 20 to apply stimulation responsively to a signal received from one or more of sensors 80. For example, responsively to the signal, the control unit may initiate stimulation, stop stimulation, increase or decrease a strength of the stimulation, and/or modify another parameter of the stimulation.

In an embodiment of the present invention, control unit 30 is configured to receive an indication of a blood glucose level sensed by a sensor 80, and to drive electrode device 20 to apply stimulation responsively to the indication. For example, the control unit may drive the electrode device to deliver the stimulation when the sensed glucose level exceeds or falls below a threshold value, and/or to modulate a level of the applied stimulation responsively to the blood glucose level.

In an embodiment of the present invention, control unit 30 is configured to receive a sensor signal indicative of ingestion of food by subject 24, and, responsively to the signal, to initiate, stop, and/or modify a parameter of the stimulation. For example, sensor 80 may comprise a blood glucose sensor, a GI motility sensor, a stomach mechanical pressure sensor, a sensor configured to detect chewing muscle activity, a gall bladder movement sensor, or a gall bladder impedance sensor.

For some applications, the control unit is configured to initiate application of the stimulation prior to the arrival of the ingested food in the duodenum. For example, the control unit may initiate the stimulation immediately upon detection of ingestion, or after a delay having a duration of less than about 10 minutes. Alternatively, if the control unit is already applying a low level of stimulation when ingestion is detected (such as in those applications in which chronic stimulation is provided, e.g., for a certain period of time each day), the control unit increases a strength of the stimulation, e.g., a frequency, amplitude, duty cycle, or other parameter(s) of the stimulation. The initiation of or increased strength of the stimulation causes a feeling of satiety that reduces the subject's desire to continue eating. In addition, the stimulation sometimes interferes with digestion of the food because the earlier than normal release of bile prevents the release of bile at the normal, optimal time for digestion.

In an embodiment of the present invention, control unit 30 initiates or increases the strength of stimulation in response to a drop in a sensed impedance of gall bladder 22. Optionally, when the impedance returns towards or to baseline, the control unit ceases or decreases the strength of the stimulation. Prior to applying stimulation, control unit 30 measures an impedance between electrode device 20 and the gall bladder, such as the gall bladder neck. The control unit increases the stimulation current responsively to a measured drop in the impedance. Such an increase may compensate for a decrease in cystic duct volume and/or an increase in the distance between the electrode device 20 and the tissue of the gall bladder. For example, if the impedance is detected to have fallen from 600 ohms at baseline to 300 ohms, the control unit may increase the current by between about 10% and about 100% of its initial value, e.g., by between about 20% and 50%, such as by about 30%. Alternatively or additionally, control unit 30 applies a fixed voltage, thereby automatically compensating for any drop in impedance.

In an embodiment of the present invention, electrode device 20 is configured to be applied to gall bladder 22 itself. Control unit 30 is configured to use at least one of the electrodes of the electrode device for both stimulating the gall bladder and sensing impedance of the gall bladder. For some applications, the configuration of the at least one electrode is similar to that of standard cardiac pacing electrodes.

For some applications, one or more of sensors 80 is configured to sense a physiological parameter not directly related to ingestion and/or digestion. For example, the sensor may comprise a heart rate sensor or a respiration sensor. For some applications, control unit 30 is configured to terminate stimulation or reduce a strength of the stimulation upon sensing of undesired effects such as bradycardia or respiratory arrest.

In an embodiment of the present invention, control unit 30 is configured to receive a signal indicative of an input, such as from subject 24 or a caregiver. For example, the control unit may be configured to allow the subject to initiate stimulation when the subject experiences a desire to eat that the subject would like to diminish without actually eating, or with eating less than the subject would otherwise expect to eat. For some applications, system 10 comprises a magnetic wand, which the subject touches to the surface of the body near the control unit in order to provide the input.

In an embodiment of the present invention, control unit 30 is configured to drive electrode device 20 to apply the stimulation according to a predefined activation schedule. For some applications, the control unit applies the stimulation at a normal expected time of eating by the subject, or a certain amount of time prior to such an expected time of eating, typically by using a clock.

In an embodiment of the present invention, electrode device 20 is configured to be placed at the gall bladder site during a laparoscopic surgical procedure. For example, when the configuration of electrode device 20 described hereinabove with reference to FIGS. 4 and 5 is used, tabs 60 may be stapled together using a laparoscopic stapling tool.

In an embodiment of the present invention, electrode device 20 is configured to be delivered endoscopically. For example, this delivery technique may be used for applications in which the electrode is configured to be placed within a duct or within gall bladder 22. For some applications, the electrode device is delivered using an endoscopic catheter to a selected location using a shape-defining electrode structure, which configures the electrode device to assume a first shape during delivery, and to assume a second (typically natural) shape after proper positioning within the duct or gall bladder.

In an embodiment of the present invention, electrode device 20 is configured to be delivered percutaneously. The electrode device is guided to the target area via a percutaneous puncture, such as by using techniques similar to those described for cholecystostomy in the above-cited article by Hong J et al. For some application, the electrode device is delivered under the guidance of ultrasound, CT, fluoroscopy (e.g., aided by oral cholecystography), MRI, or another imaging modality.

In an embodiment of the present invention, electrode device 20 is configured to be delivered using retrograde endoscopy. A catheter is advanced through the common bile duct to reach the gall bladder region, and electrode device 20 is positioned at the gall bladder site. For some applications, this approach employs techniques similar to those of Endoscopic Retrograde Cholangiopancreatography (ERCP), as is known in the art.

In an embodiment of the present invention, electrode device 20 is configured to be delivered during an open surgical procedure.

In an embodiment of the present invention, feedback is used during a placement procedure to aid with the placement of electrode device 20 at a desire location. For some applications, during the placement procedure control unit 30 configures one or more parameters of the stimulation, such as stimulation strength (e.g., frequency, amplitude, and/or duty cycle) to cause a specific patient sensation upon placement of the electrode device in the desired location. The placement location and/or stimulation parameters are manipulated to effect the desired patient sensation, in order to verify desired positioning and/or parameters. Such sensations may include, for example, a dull pain in the right side of the body, transient respiratory pain during inhalation, a feeling of mild nausea, and a feeling of satiety.

For some applications, after placement of the electrode device, one or parameters of stimulation are configured to achieve maximum strength (e.g., frequency, amplitude, and/or duty cycle) with minimal patient discomfort. Such configuration may occur during an initial calibration procedure after placement, and/or over time as the subject's body becomes accustomed to the stimulation. For example, such parameters may include the stimulation duty cycle (the timing of “on” and “off” periods), the amplitude, or the frequency of the stimulation.

For example, during a placement procedure, the location of electrode device 20 may be manipulated until a test stimulation induces the desired patient sensation. After stimulation is applied chronically (for at least one day), the stimulation “on”/“off” may be manipulated to maximize efficacy. Typically, the “on”/“off” ratio is reduced to minimize side effects, and is increased to maximize efficacy.

For some applications, stimulation of a gall bladder site as described herein prevents gall bladder stasis that is often associated with a strict diet, and thereby reduces the likelihood of the formation of gallstones. For some applications, a method is provided that comprises identifying that a subject is at risk of suffering from gallstones, such as because the subject is following a strict diet, and reducing the risk by applying stimulation to a gall bladder site.

For some applications, stimulation of a gall bladder site as described herein activates reflex circuits that increase the hepatic production of bile, thereby increasing the excretion of cholesterol and of bilirubin.

In an embodiment of the present invention, stimulation of a gall bladder site as described herein causes the reflex activation of vagal pathways, which serves as an indirect method for vagus nerve stimulation. For some applications, a site of a vagus nerve branch in a vicinity of the gall bladder (i.e., within 3 cm of the gall bladder, such as within 1 cm thereof) is stimulated, and the stimulation is configured to generate action potentials traveling in an afferent direction in the vagal nerve, so as to imitate the afferent activity associated with gall bladder contraction, generate a sensation of satiety, a reflex arc, or other feedback that treats obesity. For some applications, such stimulation is configured to be unidirectional, i.e., to substantially not generate action potentials traveling in an efferent direction towards the gall bladder. Alternatively or additionally, such stimulation is configured to selectively activate a subset of fibers of the vagus nerve based on their diameters. Such unidirectional and/or selective-fiber-size stimulation may be performed using techniques described in one or more of the applications incorporated hereinbelow by reference.

In an experiment conducted by the inventors, a cuff electrode device having an inner diameter of 4.5 mm was placed around a cystic duct of a dog near the neck of the gall bladder. A control unit drove the electrode device to apply stimulation with a frequency of 1 Hz, a pulse width of 0.4 ms, and a current of 20 mA. Stimulation by the electrode device caused immediate, substantial gastric contraction, particularly of the pyloric area of the stomach.

For some applications, any pain that may be associated with some embodiments of the present invention is reduced or eliminated using one or more of the pain-reduction techniques described in U.S. patent application Ser. No. 11/517,888, filed Sep. 7, 2006, entitled, “Techniques for reducing pain associated with nerve stimulation,” which is assigned to the assignee of the present application and is incorporated herein by reference. For some applications, the intermittent stimulation techniques described with reference to FIG. 6 of the '888 application serve to minimize any interruption of normal gall bladder activity that may be associated with some of the gall bladder stimulation techniques described herein, in addition to or instead of reducing any pain that may be associated with such gall bladder stimulation.

For some applications, embodiments of the present invention described herein comprise identifying that a subject suffers from obesity, overweight, an eating disorder, and/or diabetes, and/or that a subject could benefit from weight loss or blood glucose control, and applying the stimulation responsively to the identifying.

The term “stimulation,” as used in the present application, including the claims, is to be understood as excitation or decrease in excitation threshold of the sensory receptors of a gall bladder site and/or efferent presynaptic or postsynaptic innervation and/or excitation or decrease in excitation threshold of the muscle fibers of a gall bladder site. Such stimulation, depending on the parameters thereof and other factors, may induce a sensation, activate a reflex circuit, and/or induce the release of hormones or cause contraction or relaxation of the gall bladder.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. patent application Ser. No. 10/205,474, filed Jul. 24, 2002, entitled, “Electrode assembly for nerve control,” which published as US Patent Application Publication 2003/0050677
U.S. Provisional Patent Application 60/383,157 to Ayal et al., filed May 23, 2002, entitled, “Inverse recruitment for autonomic nerve systems”
PCT Patent Application PCT/IL02/00068, filed Jan. 23, 2002, entitled, “Treatment of disorders by unidirectional nerve stimulation,” which published as PCT Publication WO 03/018113, and U.S. patent application Ser. No. 10/488,334, filed Feb. 27, 2004, in the US National Phase thereof
U.S. patent application Ser. No. 09/944,913, filed Aug. 31, 2001, entitled, “Treatment of disorders by unidirectional nerve stimulation,” which issued as U.S. Pat. No. 6,684,105
PCT Patent Application PCT/IL03/00430, filed May 23, 2003, entitled, “Electrode assembly for nerve control,” which published as PCT Publication WO 03/099373
U.S. patent application Ser. No. 10/866,601, filed Jun. 10, 2004, entitled, “Applications of vagal stimulation,” which published as US Patent Application Publication 2005/0065553
PCT Patent Application PCT/IL04/000495, filed Jun. 10, 2004, entitled, “Applications of vagal stimulation,” which published as PCT Publication WO 04/110549
U.S. patent application Ser. No. 11/022,011, filed Dec. 22, 2004, entitled, “Construction of electrode assembly for nerve control,” which published as US Patent Application Publication 2006/0136024
U.S. patent application Ser. No. 11/062,324, filed Feb. 18, 2005, entitled, “Techniques for applying, calibrating, and controlling nerve fiber stimulation,” which published as US Patent Application Publication 2005/0197675
U.S. patent application Ser. No. 11/064,446, filed Feb. 22, 2005, entitled, “Techniques for applying, configuring, and coordinating nerve fiber stimulation,” which published as US Patent Application Publication 2005/0267542
U.S. patent application Ser. No. 11/280,884, filed Nov. 15, 2005, entitled, “Techniques for nerve stimulation,” which published as US Patent Application Publication 2006/0106441
U.S. patent application Ser. No. 10/745,514, filed Dec. 29, 2003, entitled, “Nerve-branch-specific action-potential activation, inhibition, and monitoring,” which published as US Patent Application Publication 2005/0149154
U.S. patent application Ser. No. 11/234,877, filed Sep. 22, 2005, entitled, “Selective nerve fiber stimulation,” which published as US Patent Application Publication 2006/0100668
U.S. patent application Ser. No. 11/517,888, filed Sep. 7, 2006, entitled, “Techniques for reducing pain associated with nerve stimulation”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating a patient, comprising:
an electrode device, configured to be placed in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port; and
a control unit, configured to drive the electrode device to apply a current to the site, and to configure the current to induce weight loss of the patient.

2. (canceled)

3. The apparatus according to claim 1, wherein the gall bladder site includes at least one site selected from the group consisting of: the neck of the gall bladder, and the cystic duct, and wherein the electrode device is configured to be placed in the vicinity of the selected site.

4-5. (canceled)

6. The apparatus according to claim 1, wherein the control unit is configured to drive the electrode device to apply the current responsively to an indication of eating of the patient.

7-12. (canceled)

13. The apparatus according to claim 1, wherein the electrode device comprises at least one electrode and a cuff coupled to the at least one electrode, and wherein the cuff is configured to be placed around at least 270 degrees of the at least one site.

14. The apparatus according to claim 1, wherein the control unit is configured to configure the current to cause at least one effect selected from the group consisting of: gastric contraction, and delayed gastric emptying.

15. The apparatus according to claim 1, wherein the electrode device is configured to be fully implanted in a body of the patient.

16. The apparatus according to claim 1, wherein the control unit is configured to configure the current to activate nervous tissue near or at the gall bladder site.

17. (canceled)

18. The apparatus according to claim 1, wherein the control unit is configured to induce the weight loss by inducing a feeling of satiety in the patient by driving the electrode device to apply the current.

19. (canceled)

20. The apparatus according to claim 18, wherein the control unit is configured to induce the feeling of satiety by configuring the current to disturb naturally-occurring organized rhythmic activity of a stomach of the patient.

21. (canceled)

22. The apparatus according to claim 1, wherein the control unit is configured to induce the weight loss by causing at least one effect by driving the electrode device to apply the current, the at least one effect selected from the group consisting of: a reduced absorption of ingested food, increased peristaltic motion of one or more organs of a gastrointestinal tract of the patient, and decreased transit time of food through a duodenum.

23. (canceled)

24. The apparatus according to claim 1, wherein the control unit is configured to configure the current to cause contraction of a stomach of the patient.

25-26. (canceled)

27. Apparatus for treating a patient, comprising:
a coupling element, configured to be placed around at least 270 degrees of at least one site of the patient selected from the group consisting of: a neck of a gall bladder, a cystic duct, a hepatic duct, a common hepatic duct, a bile duct, and a common bile duct; and
at least one electrode, coupled to the coupling element such that the at least one electrode is positioned in a vicinity of the at least one site when the coupling element is coupled to the at least one site.

28-29. (canceled)

30. The apparatus according to claim 27, wherein the at least one site includes at least one site selected from the group consisting of: the neck of the gall bladder, and the cystic duct, and wherein the coupling element is configured to be placed around at least 270 degrees of the selected site.

31. The apparatus according to claim 27, comprising a control unit, configured to drive the at least one electrode to apply a current to the at least one site.

32. The apparatus according to claim 31, wherein the control unit is configured to configure the current to stimulate nervous tissue in a vicinity of the at least one site.

33. (canceled)

34. The apparatus according to claim 31, wherein the control unit is configured to configure the current to cause contraction of a stomach of the patient.

35-39. (canceled)

40. The apparatus according to claim 31, wherein the control unit is configured to configure the current to induce weight loss of the patient.

41. The apparatus according to claim 40, wherein the control unit is configured to induce the weight loss by inducing a feeling of satiety in the patient by driving the at least one electrode to apply the current.

42. The apparatus according to claim 31, wherein the control unit is configured to configure the current to treat diabetes of the patient.

43. (canceled)

44. The apparatus according to claim 31, wherein the control unit is configured to drive the at least one electrode to apply the current responsively to an indication of eating of the patient.

45. The apparatus according to claim 27, wherein the coupling element comprises a cuff, which is configured to be placed around at least 270 degrees of the at least one site.

46-113. (canceled)

114. A method for treating a patient, comprising:
identifying that the patient may benefit from weight loss; and
responsively to the identifying, inducing the weight loss by applying electrical stimulation to at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port.

115. (canceled)

116. The method according to claim 114, wherein applying the stimulation comprises applying the stimulation to at least one site selected from the group consisting of: the neck of the gall bladder, and the cystic duct.

117. (canceled)

119. The method according to claim 114, wherein applying the stimulation comprises applying the stimulation responsively to an indication of eating of the patient.

120-121. (canceled)

122. The method according to claim 114, wherein applying the stimulation comprises configuring the stimulation to cause at least one effect selected from the group consisting of: gastric contraction, and delayed gastric emptying.

123-126. (canceled)

127. The method according to claim 114, wherein applying the stimulation comprises fully implanting an electrical stimulator in a body of the patient, and applying the stimulation using the electrical stimulator.

128. The method according to claim 114, wherein applying the stimulation comprises identifying that the electrical stimulation is suitable for activating nervous tissue in a vicinity of the gall bladder site.

129. (canceled)

130. The method according to claim 114, wherein inducing the weight loss comprises inducing a feeling of satiety in the patient by applying the stimulation.

131. The method according to claim 130, wherein inducing the feeling of satiety comprises configuring the stimulation to cause at least one effect selected from the group consisting of: gastric contraction, and delayed gastric emptying.

132. The method according to claim 130, wherein inducing the feeling of satiety comprises configuring the stimulation to disturb naturally occurring organized rhythmic activity of a stomach of the patient.

133. (canceled)

134. The method according to claim 114, wherein inducing the weight loss comprises causing at least one effect by applying the stimulation, the at least one effect selected from the group consisting of: a reduced absorption of ingested food, increased peristaltic motion of one or more organs of a gastrointestinal tract of the patient, and decreased transit time of food through a duodenum.

135. (canceled)

136. The method according to claim 114, wherein applying the stimulation comprises configuring the stimulation to cause contraction of a stomach of the patient.

137. (canceled)

138. (canceled)

139. A method for treating a patient, comprising:
placing a coupling element around at least 270 degrees of at least one site of the patient selected from the group consisting of: a neck of a gall bladder, a cystic duct, a hepatic duct, a common hepatic duct, a bile duct, and a common bile duct, such that at least one electrode coupled to the coupling element is positioned in a vicinity of the at least one site; and
applying an electrical current to the at least one site using the at least one electrode.

140. The method according to claim 139, wherein the at least one site includes at least one site selected from the group consisting of: the neck of the gall bladder, and the cystic duct, and wherein placing the coupling element comprises placing the coupling element around at least 270 degrees of the selected site.

141. The method according to claim 139, wherein applying the current comprises configuring the current to stimulate nervous tissue in a vicinity of at least one site.

142. The method according to claim 139, wherein applying the current comprises configuring the current to cause contraction of a stomach of the patient.

143. The method according to claim 139, wherein applying the current comprises configuring the current to treat diabetes of the patient.

144. The method according to claim 139, wherein applying comprises applying the current responsively to an indication of eating of the patient.

145. The method according to claim 139, wherein applying the current comprises configuring the current to induce weight loss of the patient.

146. The method according to claim 145, wherein configuring the current to induce the weight loss comprises inducing a feeling of satiety in the patient by configuring the current.

147-151. (canceled)

152. A method for treating a patient, comprising:
identifying an electrical signal as being suitable for activating nervous tissue; and
applying the identified signal in a vicinity of at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port.

153. The method according to claim 152, wherein applying the identified signal comprises applying the identified signal in the vicinity of at least one site selected from the group consisting of: the neck of the gall bladder, and the cystic duct.

154-157. (canceled)

158. A method for treating a patient, comprising:
identifying that the patient suffers from diabetes; and
responsively to the identifying, treating the diabetes by applying electrical stimulation to at least one gall bladder site of the patient selected from the group consisting of: a gall bladder, a neck of the gall bladder, a cystic duct, a hepatic duct, a common hepatic duct, a bile duct, a common bile duct, sensory fibers of the gall bladder, and a hepatic port.

159. (canceled)

160. The method according to claim 158, wherein applying the stimulation comprises applying the stimulation to at least one site selected from the group consisting of: the neck of the gall bladder, and the cystic duct.

161-169. (canceled)
